(19) United States
(12) Patent Application Publication
(10) Pub. No.: US 2014/0322281 A1
(43) Pub. Date: Oct. 30, 2014

(54) LIQUID COSMETIC

(71) Applicant: MITSUBISHI PENCIL COMPANY, LIMITED, Shinagawa-ku (JP)

(72) Inventor: Hiroshi SATOU, Fujioka-shi (JP)

(73) Assignee: MITSUBISHI PENCIL COMPANY, LIMITED, Shinagawa-ku (JP)

(21) Appl. No.: 14/257,327

(22) Filed: Apr. 21, 2014

(30) Foreign Application Priority Data
Apr. 24, 2013 (JP) 2013-091056

(51) Int. Cl. A61K 8/19; A61Q 1/10; A61K 8/02

(52) U.S. Cl. CPC A61K 8/19; A61K 8/0245; A61Q 1/10
USPC 424/401; 424/63

(57) ABSTRACT

Provided is a liquid cosmetic which is stored in a liquid cosmetic applicator using a brush or a pen feed as an applying means and which is excellent in a dispersion stability of black iron oxide used as a black pigment, a hue of the black color and a discharge performance. The above liquid cosmetic comprises at least 1 to 30% by mass of black iron oxide, water, 0.5 to 5% by mass of a dispersant comprising a film-formable resin, 2 to 15% by mass (in terms of a solid content) of a film-forming agent, 0.1 to 3% by mass of a surfactant, 0.05 to 5% by mass of volatile alcohol, and 5 to 20% by mass of glycols, wherein a viscosity thereof measured at a temperature of 25° C. and a shear rate of 3.83 s⁻¹ by means of an ELD type viscometer falls in a range of 2 to 10 mPa·s; and an observed average particle diameter is 100 to 300 nm.

This Nonprovisional application claims priority under 35 U.S.C. §119(a) on Patent Application No. 2013-91056 filed in Japan on 24 Apr. 2013, the entire contents of which are hereby incorporated by reference.

### BACKGROUND ART OF THE INVENTION

1. Field of the Invention

The present invention relates to a liquid cosmetic, more specifically to a liquid cosmetic which is suited to a sliver type vessel holding a liquid cosmetic in a sliver or a collector type vessel holding a liquid cosmetic in a flat-spread sheet member. Each vessel has an applying part using a brush or a pen feed as an applying means, wherein the liquid cosmetic is prepared by using black iron oxide as a black pigment and is excellent in a discharge performance.

2. Description of the Related Art

Cosmetics prepared by dissolving dyes as a colorant in water, water-soluble organic solvents or the like and cosmetics prepared by dispersing pigments in water, water-soluble organic solvents or the like with surfactants and water-soluble resins have so far been known as liquid cosmetics of such a type as stored in an applicator. The above conventional liquid cosmetics are unsatisfactory in a coloring power when a dye is used. Also, carbon blacks used as a black pigment are not permitted in some countries to use carbon blacks as a colorant for cosmetics, and carbon blacks are restrictively used in some country.

On the other hand, having a magnetism, black iron oxide readily aggregates and is difficult to disperse and, pen feeds, joining feeds and slivers are clogged to inhibit a content liquid from being fed to pen tips due to the involved many coarse particles.

Known as conventional liquid cosmetics prepared by using black iron oxide as a black pigment are, for example, 1) an eyeliner filled in a pen type vessel which comprises black iron oxide, red iron oxide, Prussian blue, an anionic dispersant and a nonionic dispersant (refer to, for example, patent document 1), 2) a liquid cosmetic comprising at least 75% by weight or more of black iron oxide having a particle diameter of 1.2 μm or less as a colorant, a substance such as sodium thiosulfate, glucose and ascorbic acid having a reducing property so as to prevent the above black iron oxide from discoloring by inhibiting the oxidation, and a solvent (refer to, for example, patent document 2) and 3) a makeup cosmetic characterized by blending tabular black iron oxide having an average particle diameter of 5 to 100 μm and a thickness of 0.2 to 3 μm or tabular black iron oxide modified to hydrophobic (refer to, for example, patent document 3).

In patent document 1 described above, however, it is described that black iron oxide, red iron oxide and Prussian blue are essential three components and that blending of black iron oxide alone makes it difficult to disperse the components even by blending the anionic dispersant and the nonionic dispersant and makes it impossible to disperse the components sufficiently well and stabilize the dispersion. The above descriptions are supported in Comparative Example 3, and the existing situation is that a problem still remains in a dispersion stability thereof when only black iron oxide is used as a black pigment.

The respective liquid cosmetics shown in patent documents 2 and 3 described above are still unsatisfactory in terms of a dispersion stability of black iron oxide used as a black pigment after ageing and a hue of the cosmetics.

Patent document 1: JP 2003-231614A (claims, examples and others)
Patent document 2: JP Hei 3-7213A (claims, examples and others)
Patent document 3: JP 2010-222295A (claims, examples and others)

### SUMMARY OF THE INVENTION

In light of the foregoing problems and existing situations of the conventional art, the present invention intends to solve the problems, and an object thereof is to provide a liquid cosmetic which is suited to a sliver type vessel holding a liquid cosmetic in a sliver or a collector type vessel holding a liquid cosmetic in a flat-spread sheet member. Each vessel has an applying part using a brush or a pen feed as an applying means, wherein the liquid cosmetic is excellent in a dispersion stability of black iron oxide used as a black pigment, a hue of the black color and a discharge performance.

In light of the problems on the conventional art described above and the like, intense investigations repeated by the present inventors have resulted in finding that a liquid cosmetic which meets the object described above is provided by a liquid cosmetic stored in a liquid cosmetic applicator using a brush or a pen feed as an applying means, wherein the above liquid cosmetic comprises at least black iron oxide having a specific particle diameter, water, a specific content of a dispersant comprising a film-formable resin, a surfactant, and the like, and a viscosity of the liquid cosmetic is controlled in a specific range. Thus, the present invention has come to be completed.

That is, the present invention comprises the following items (1) to (7).

(1) A liquid cosmetic stored in a liquid cosmetic applicator using a brush or a pen feed as an applying means, wherein the above liquid cosmetic comprises at least 1 to 30% by mass of black iron oxide, water, 0.5 to 5% by mass of a dispersant comprising a film-formable resin, 2 to 15% by mass as a solid content of a film-forming agent, 0.1 to 3% by mass of a surfactant, 0.05 to 5% by mass of volatile alcohol, and 5 to 20% by mass of glycols; a viscosity thereof measured at a temperature of 25° C. and a shear rate of 3.83 s⁻¹ by means of an ELD type viscometer falls in a range of 2 to 10 mPa·s; and an observed average particle diameter is 100 to 300 nm.
(2) The liquid cosmetic as described in the above item (1), wherein the dispersant comprising a film-formable resin is a homopolymer or a copolymer prepared by using one kind or two or more kinds of compounds selected from acrylic acid, methacrylic acid or alkyl esters thereof as a starting material monomer, or a copolymer of at least one of acrylic acid, methacrylic acid or alkyl esters thereof with octylacrylamide.
(3) The liquid cosmetic as described in the above item (1), wherein the surfactant is a nonionic surfactant.
(4) The liquid cosmetic as described in the above item (1), wherein the volatile alcohol is phenoxyethanol.
(5) The liquid cosmetic as described in the above item (1), wherein the glycols are 1,3-butylene glycol.
(6) The liquid cosmetic as described in the above item (1), wherein the liquid cosmetic applicator is provided with a sliver type vessel in which the liquid cosmetic is held in a sliver or a collector type vessel in which the liquid cosmetic is held in a flat-spread sheet member.
(7) The liquid cosmetic as described in the above item (1), wherein the liquid cosmetic is used for an eyeliner or an eyebrow.

According to the present invention, provided is a liquid cosmetic which can be used for a liquid cosmetic applicator using a brush or a pen feed as an applying means and which is excellent in a dispersion stability of black iron oxide used as a black pigment, a hue of the black color and a discharge performance.

### BRIEF DESCRIPTION OF THE DRAWINGS

FIG. 1 is a partial cross section showing one example of the embodiment of the liquid cosmetic of the present invention.

FIG. 2 is a partial cross section showing another example of the embodiment of the liquid cosmetic of the present invention.

### DETAILED DESCRIPTION OF THE PREFERRED EMBODIMENTS

The embodiments of the present invention shall be explained below in detail.

The liquid cosmetic of the present invention is characterized by that it is a liquid cosmetic stored in a liquid cosmetic applicator using a brush or a pen feed as an applying means, wherein the above liquid cosmetic comprises at least 1 to 30% by mass of black iron oxide, water, 0.5 to 5% by mass of a dispersant comprising a film-formable resin, 2 to 15% by mass (in terms of a solid content) of a film-forming agent, 0.1 to 3% by mass of a surfactant, 0.05 to 5% by mass of volatile alcohol, and 5 to 20% by mass of glycols; a viscosity thereof measured at a temperature of 25° C. and a shear rate of 3.83 s⁻¹ by means of an ELD type viscometer falls in a range of 2 to 10 mPa·s; and an observed average particle diameter is 100 to 300 nm.

The black iron oxide (Fe₃O₄) used in the present invention is used as a black pigment (colorant), and the product having a particle size in which an observed average particle diameter is 100 to 300 nm is used.

The particle form of the black iron oxide shall not specifically be restricted as long as the black iron oxide has the above particle size, and the products comprising preferably spherical particles, octahedral particles and the like can be used. The “black iron oxide dispersion” referred to in the present invention means a black iron oxide dispersion (liquid cosmetic) obtained by blending the black iron oxide with a dispersant comprising a film-formable resin, a surfactant, volatile alcohol, glycols, water and the like which are described later.

In the present invention, a particle size of the black iron oxide in the dispersion is controlled owing to a suitable blend composition of the black iron oxide with a dispersant comprising a film-formable resin, a surfactant, volatile alcohol, glycols, water and the like which are described later, whereby the black iron oxide is prevented from aggregation in water to make it possible to provide high dispersibility thereof; the liquid cosmetic is provided with a satisfactory hue; and the liquid cosmetic does not cause clogging in a pen feed, a joining feed and a sliver and can be provided with an excellent discharge performance.

An observed average particle diameter of the liquid cosmetic is preferably 100 nm or more from the viewpoint of bleeding into a texture and wrinkles on the skin. Also, the observed average particle diameter is preferably 300 nm or less from the viewpoint of settling of the pigment and clogging in a pen feed and a joining feed. In this connection, the “average particle diameter” referred to in the present invention is a value determined at 25° C. by means of a particle diameter measuring instrument FPAR-1000 (manufactured by Otsuka Electronics Co., Ltd.) according to a dynamic light scattering method.

A content of the black iron oxide having the above characteristics is preferably 1 to 30% by mass (hereinafter referred to merely as %) based on a total amount of the liquid cosmetic, more preferably 5 to 25%. If a content of the black iron oxide is less than 1%, the developed color is too pale and unsatisfactory for the cosmetic. On the other hand, if the content of the black iron oxide exceeds 30%, the liquid cosmetic is increased in a viscosity too much and is not smoothly discharged through the liquid cosmetic applicator of a sliver type or a collector type used in the present invention. Accordingly, both are undesirable.

The dispersant used in the present invention comprises a film-formable resin, and the dispersant enhances further dispersibility of the black iron oxide having the particle size described above and also functions as a resin for forming a film.

The dispersant which can be used shall not specifically be restricted as long as the dispersant has the function described above, and used is, for example, a homopolymer or a copolymer prepared by using one kind or two or more kinds of compounds selected from acrylic acid, methacrylic acid or alkyl esters thereof as a starting material monomer, or a copolymer of at least one of acrylic acid, methacrylic acid or alkyl esters thereof with octylacrylamide. The homopolymer or the copolymer prepared by using one kind or two or more kinds of the compounds selected from acrylic acid, methacrylic acid or alkyl esters thereof as a starting material monomer is preferably an acrylic acid and alkyl acrylate copolymer which has an acidic residue as a side chain in a repetitive structure and which can be dissolved into water by neutralization. The particularly preferred alkyl acrylate copolymer includes a copolymer comprising tert-butyl acrylate, ethyl acrylate and methacrylic acid.

A content of the above dispersant is preferably 0.5 to 5%, more preferably 2 to 4% based on a total amount of the liquid cosmetic.

If a content of the above dispersant is less than 0.5%, a dispersion stability of the black iron oxide is unsatisfactory. On the other hand, if the dispersant is contained in an amount exceeding 5%, the viscosity is increased too much, and the dispersion stability is not enhanced, so that it is not economical.

The film-forming agent used in the present invention includes, for example, emulsion resins of copolymers comprising monomers selected from one kind or two or more kinds of acrylic acid, methacrylic acid, alkyl esters or derivatives thereof, styrene and vinyl acetate.

In the present invention, the dispersant described above comprises as well a film-formable resin. The dispersant is different from the film-forming agent in terms of a soluble resin or an emulsion resin. The emulsion resin is an aqueous suspension of a polymer obtained by an emulsion polymerization of a monomer in water as a polymerization solvent. For dispersion of the black iron oxide in the present invention, a more stable aqueous dispersion of the black iron oxide is obtained in the soluble resin rather than in the emulsion resin. Thus, the dispersant and the film-forming agent are used in distinction from others by taking the above point into consideration.

A content of the film-forming agent of emulsion resin is preferably 2 to 15%, more preferably 2 to 10% in terms of a solid resin content based on a total amount of the liquid cosmetic.

If a content of the above film-forming agent of emulsion resin is less than 2% in terms of a solid resin content, the water resistant performance is deteriorated. On the other hand, if the film-forming agent of is contained in an amount exceeding 15%, an applying part of the liquid cosmetic applicator such as brush and pen feed is dried, and the problem that the liquid cosmetic cannot be applied occurs in a certain case. Accordingly, both are undesirable.

The surfactant used in the present invention functions as a dispersion auxiliary agent for dispersing the black iron oxide, and the surfactant shall not specifically be restricted as long as the surfactant is a nonionic surfactant applicable to cosmetics. Preferably used are surfactants which are nonionic surfactants of a polyoxyalkylene adduct type of polyoxyethylene (hereinafter referred to as POE), polyoxypropylene (hereinafter referred to as POP) and the like and which are modified to hydrophilic.

For example, POE alkyl ethers such as POE lauryl ether, POE cetyl ether, POE stearyl ether, POE oleyl ether, POE behenyl ether, POE 2-octyldodecyl ether, POE 2-hexyldecyl ether, and POE isostearyl ether, POE·POP alkyl ethers such as POE·POP cetyl ether and POE·POP decyltetradecyl ether, POE glycerin fatty acid esters such as POE hydrogenated castor oil, POE castor oil, POE glyceryl monostearate, POE glyceryl monooleate, and POE glyceryl monoisostearate, POE sorbitan fatty acid esters such as POE sorbitan monostearate and POE sorbitan monooleate, POE sorbitol fatty acid esters such as POE sorbitol monolaurate, polyethylene glycol (PEG) fatty acid esters such a PEG monostearate, PEG monooleate, and PEG monolaurate, which are modified to hydrophilic by controlling the addition molar numbers of POE and POP, can be listed. In particular, the POE alkyl ethers are preferable. The nonionic surfactants can be used alone or in combination of two or more kinds thereof.

A content of the above surfactants is preferably 0.1 to 3%, more preferably 0.1 to 2% based on a total amount of the liquid cosmetic.

If a content of the above dispersant is less than 0.1% by mass, a dispersion stability of the black iron oxide is unsatisfactory. If the content of the above dispersant exceeds 3%, a water resistance of the liquid cosmetic is unsatisfactory, and an adhesive force thereof to the skin is too weak. Accordingly, both are undesirable.

The volatile alcohol used in the present invention shall not specifically be restricted as long as the volatile alcohol is usually used for cosmetics, and any products can be used. The volatile alcohol includes, for example, ethanol, 2-propanol, phenoxyethanol and the like. The above alcohols are used usually for cosmetics as a cooling agent, a solvent and an antimicrobial agent, and it has been found that such alcohols can prevent the pigment of the black iron oxide from being settled down and aggregation only when such alcohols are blended in combination as is the case with the present invention and that a liquid cosmetic which has a smooth drawing feeling and which is suited to an aqueous eyeliner and the like is obtained. Thus, the volatile alcohol is used in the present invention for a quite different purpose from conventional use.

In the present invention, the above compounds may be used alone or can be used in combination of two or more kinds thereof, and phenoxyethanol is most preferably used.

A content of the above volatile alcohols is preferably 0.05 to 5%, more preferably 0.1 to 3% based on a total amount of the liquid cosmetic. A storage stability of the liquid cosmetic is particularly good in the above ranges.

The glycols used in the present invention shall not specifically be restricted as long as the glycols are usually used for cosmetics, and any products can be used. The glycols include, for example, glycol, propylene glycol, dipropylene glycol, 1,3-butylene glycol, 1,2-pentylene glycol and the like. The above compounds are used usually for cosmetics as a moisturizing agent and an antimicrobial agent, and it has been found that such glycols can prevent the pigment of the black iron oxide from being settled down and aggregation only when such glycols are blended in combination as is the case with the present invention and that a liquid cosmetic which has a smooth drawing feeling and which is suited to an aqueous eyeliner and the like is obtained. Thus, the glycols are used in the present invention for a quite different purpose from conventional use.

The above glycols may be used alone or can be used in combination of two or more kinds thereof, and 1,3-butylene glycol is most preferably used.

A content of the above glycols is 5 to 20%, preferably 5 to 15% based on a total amount of the liquid cosmetic. A storage stability of the liquid cosmetic is particularly good in the above ranges.

In the liquid cosmetic of the present invention, water (including refined water, distilled water, ion-exchanged water, purified water, ultrapure water and the like) is used as a solvent. A content of water is a balance obtained by deducting the amounts of the respective components described above and optional components described later from a total amount of the liquid cosmetic. The liquid cosmetic of the present invention is an aqueous liquid cosmetic, and therefore a content of water is preferably 60% or more.

Further, optional components used for conventional liquid cosmetics in addition to the respective essential components described above can be contained in the liquid cosmetic of the present invention. To be specific, preservatives, antioxidants, neutralizing agents, UV absorbers, chelating agents, moisturizers, beauty ingredients, fragrances, viscosity modifiers and the like can be contained in suitable amounts as long as the effects of the present invention are not damaged.

For the liquid cosmetic of the present invention, a viscosity thereof measured at a temperature of 25° C. and a shear rate of 3.83 s⁻¹ by means of an ELD type viscometer falls in a range of 2 to 10 mPa·s, particularly preferably 3 to 6 mPa·s.

If the above viscosity value is less than 2 mPa·s, the liquid flows into wrinkles and the like to cause feathering. On the other hand, if the above viscosity exceeds 10 mPa·s, it is too viscose to be discharged smoothly for the liquid cosmetic stored in the liquid cosmetic applicator using a brush or a pen feed as an applying means in the present invention, so that such a high viscosity is undesirable. The above viscosity means a value measured on conditions, to be specific, measured at a temperature of 25° C. and a shear rate of 3.83 s⁻¹ in 1 rpm of a standard corn rotor by means of an ELD type viscometer manufacture by Tokimec Inc. including examples and others described later. The viscosity can be modified to the ranges described above by suitably combining the amounts of the respective components of the black iron oxide, water, the dispersant comprising a film-formable resin, the film-forming agent, the surfactant, the volatile alcohol and the glycols.

The liquid cosmetic of the present invention falling in the foregoing ranges of a viscosity and a particle diameter can be prepared by subjecting the black iron oxide, water, the dispersant comprising a film-formable resin, the film-forming agent, the surfactant, the volatile alcohol and the glycols to wet crushing treatment by means of a media mill.

In the present invention, the liquid cosmetic applicator using a brush or a pen feed as an applying means shall not specifically be restricted as long as the applicator is a liquid cosmetic applicator equipped with a brush or a pen feed which is used for an eyeliner or an eyebrow. Preferably an applicator which is excellent in usability, a convenience and an applying property and which is provided with a sliver type vessel holding a liquid cosmetic in a sliver or a collector type vessel holding a liquid cosmetic in a flat-spread sheet member, is suggested.

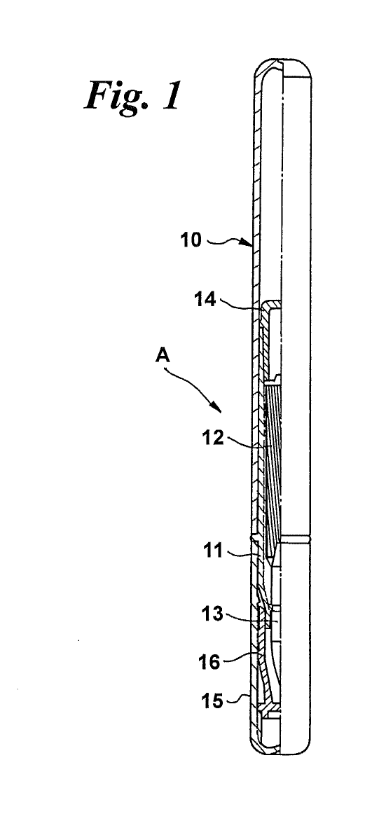

The liquid cosmetic applicator of a sliver type includes, for example, an applicator having a structure in which, as shown in FIG. 1, an inner holder 11 is provided in an applicator main body 10; an impregnation member 12 comprising a sliver and the like which is impregnated with a liquid cosmetic is received in the above inner holder 11; an applying member 13 for applying the liquid cosmetic is provided at a tip side of the impregnation member 12; and a plug 14 is fixed at a rear end of the inner holder 11. A member 15 is a cap member having an inner cap part 16. The liquid cosmetic applicator A of the above embodiment is used by removing the cap 15.

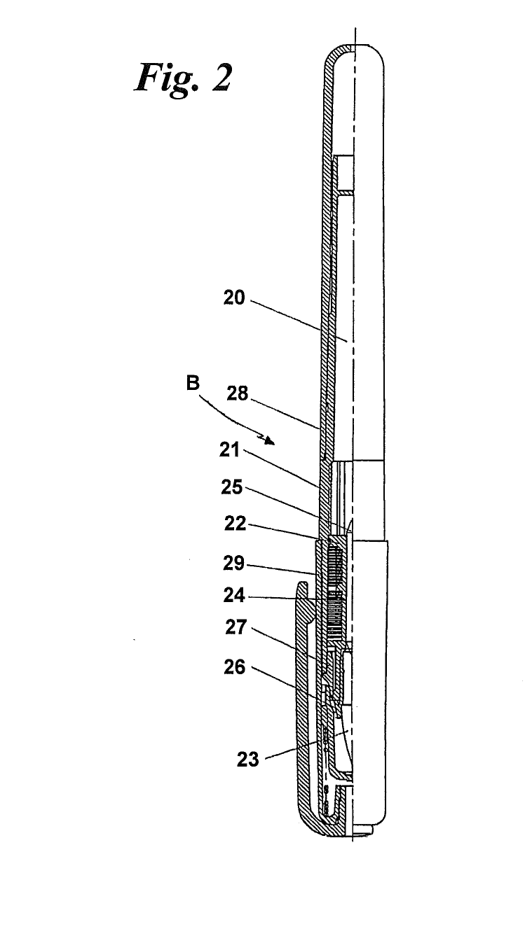

The liquid cosmetic applicator of a collector type of a free liquid system includes, for example, an applicator in which, as shown in FIG. 2, a liquid cosmetic 20 is filled in a reservoir part 21 as a holder storing the free liquid cosmetic without being absorbed into sliver and the like. Provided is a constitution in which a flat-spread sheet member 22 that is a reservoir or a collector member for temporarily holding the liquid cosmetic 20 pushed out from the reservoir part 21 in order to prevent the liquid cosmetic 20 from dripping through a pen tip and an air vent when air in the reservoir part 21 is expanded by a rise in temperature is built in a front part of the reservoir part 21 and in which a pen tip (brush) 23 of a brush type as an applying member is provided in a tip part of the collector member 22.

The liquid cosmetic is delivered from the reservoir part 21 to the pen tip 23 by delivering the liquid cosmetic 20 from the reservoir part 21 to the pen tip 23 via a relay feed 25 provided with an passage 24 set in a center of the collector member 22.

Members 26, 27 in FIG. 2 are holder members, and a member 28 is a rear holder fixed in a rear part of the reservoir part 21. A member 29 is a cap part having an inner cap. Also, the liquid cosmetic may be delivered by arranging a rear part of the pen tip 23 directly in the reservoir part 21 without interposing the relay feed 25 therebetween. The liquid cosmetic applicator B of the above embodiment is used by removing the cap 29.

The liquid cosmetic of the present invention thus constituted is a liquid cosmetic stored in a liquid cosmetic applicator using a brush or a pen feed as an applying means, wherein the above liquid cosmetic comprises at least 1 to 30% by mass of the black iron oxide, water, 0.5 to 5% by mass of the dispersant comprising a film-formable resin, 2 to 15% by mass (in terms of a solid content) of the film-forming agent, 0.1 to 3% by mass of the surfactant, 0.05 to 5% by mass of the volatile alcohol and 5 to 20% by mass of the glycols; a viscosity thereof measured at a temperature of 25° C. and a shear rate of 3.83 s⁻¹ by means of an ELD type viscometer falls in a range of 2 to 10 mPa·s; an observed average particle diameter falls in a range of 100 to 300 nm. Accordingly, provided the specific effect that obtained is the liquid cosmetic which is excellent in a dispersion stability of the black iron oxide used as a black pigment, a hue of the black color and a discharge performance. Furthermore, above liquid cosmetic makes it easy to draw fine lines and is suitable for use in a black color base eyeliner or eyebrow of a low viscosity using black iron oxide as a colorant.

### EXAMPLES

Next, the present invention shall be explained in further details with reference to examples and comparative examples, but the present invention shall not be restricted to the examples shown below.

### Examples 1 to 10 and Comparative Examples 1 to 11

Liquid cosmetics (blend unit: % by mass, total amount: 100% by mass) having component type shown in the following Table 1 and Table 2 were prepared by subjecting the components to wet crushing treatment by means of a media mill and mixing and stirring them by means of a disperser, and the liquid cosmetics thus obtained were used to measure the viscosity values and the particle diameters of the respective liquid cosmetics by the measuring methods described above and evaluate a adhesion property, an applying performance and a content liquid stability thereof and a restirring property of the precipitated pigment by the following respective evaluating methods.

The results thereof are shown in the following Table 1 and Table 2.

### Evaluation Method of Adhesion Property:

The respective liquid cosmetics were filled in a cosmetic vessel of a brush pen type shown in FIG. 2, and the liquid cosmetic was applied on a back of a hand and dried for 10 minutes. Then, the applied cosmetic was exposed water flow and rubbed with a finger cushion to evaluate a removed degree of the applied cosmetic by visually as a sensory evaluation according to the following evaluation criteria.

### Evaluation Criteria:

⊚: very good (the applied part is not peeled off at all, very good)
◯: good (the applied part is slightly peeled off, good)
Δ: average (the applied part is partially peeled off)
X: inferior (the applied part is almost peeled off)

### Evaluation Method of Applying Performance:

The respective liquid cosmetics were filled in the cosmetic vessel of a brush pen type shown in FIG. 2. Five lines having a width of 1 to 2 mm and a length of about 5 cm were drawn on a back of a hand to evaluate a drawn state and a drawn line intensity thereof according to the following evaluation criteria.

### Evaluation Criteria:

⊚: drawn lines are intense and easy to draw
◯: easy to draw and sufficiently intense
Δ: slight starving and feathering are observed, but practically acceptable
X: starving and feathering are observed, and unacceptable Evaluation method of content liquid stability:

The respective liquid cosmetics as content liquids were put in a vessel with a lid and kept in a thermostat chamber at 50° C. for 1 month, and then a viscosity thereof was measured. The viscosity was compared with an initial viscosity thereof to evaluate stability thereof according to the following evaluation criteria.

### Evaluation Criteria:

◯: difference from the initial value is ±1 (mPa·s) or less
Δ: difference from the initial value is between ±1 (mPa·s) and ±2 (mPa·s)
X: difference from the initial value exceeds ±2 (mPa·s)

### Evaluation Method of Restirring Property of Precipitated Pigment:

A vial bottled was filled with the content liquid, and a stirring ball made of stainless steel was put therein. The bottled was kept in a thermostat chamber at 50° C. for 1 month, and the presence of the precipitated pigment and hardness thereof were evaluated by mobility of the stirring ball according to the following evaluation criteria.

### Evaluation Criteria:

◯: no pigment is precipitated, and the stirring ball moves smoothly by stirring once
Δ: pigment is precipitated, as a soft cake, and therefore the stirring ball moves by stirring twice to three times
X: precipitated pigment is turned into a hard cake, and the stirring ball does not move even by stirring three times

TABLE 1
(total amount: 100% by mass)

| | | Example | | | | | | | | | |
|---|---|---|---|---|---|---|---|---|---|---|---|
| Component type | Specific compound name | 1 | 2 | 3 | 4 | 5 | 6 | 7 | 8 | 9 | 10 |
| Pigment | Black iron oxide (spherical particle) | 10 | | 10 | 10 | 10 | 10 | 10 | 10 | 10 | 10 |
| Pigment | Black iron oxide (octahedral particle) | | 10 | | | | | | | | |
| Dispersant | Alkyl acrylate copolymer | 3 | 3 | | 3 | 0.5 | 5 | 3 | 3 | 3 | 3 |
| Dispersant | Octylacrylamide•acrylate copolymer | | | 3 | | | | | | | |
| Surfactant | Polyoxyethylene behenyl ether | 2 | 2 | 2 | | 2 | 2 | 0.1 | 3 | 2 | 2 |
| Surfactant | Polyethylene glycol monostearate | | | | 2 | | | | | | |
| Film-forming agent emulsion | Alkyl acrylate copolymer | 20 | 20 | 20 | 20 | 20 | 20 | 5 | 35 | 20 | 20 |
| (resin content in emulsion) | Emulsion *1 | 8.4 | 8.4 | 8.4 | 8.4 | 8.4 | 8.4 | 2.1 | 14.7 | 8.4 | 8.4 |
| Neutralizer | Aminomethylpropanol | 0.3 | 0.3 | 0.3 | 0.3 | 0.3 | 0.3 | 0.3 | 0.3 | 0.3 | 0.3 |
| Volatile alcohol | Phenoxyethanol | 0.4 | 0.4 | 0.4 | 0.4 | 0.4 | 0.4 | 0.4 | 0.4 | 0.05 | 3 |
| Glycols | 1,3-Butylene glycol | 12 | 12 | 12 | 12 | 12 | 12 | 12 | 7 | 5 | 15 |
| Preservative | Methylparaben | 0.3 | 0.3 | 0.3 | 0.3 | 0.3 | 0.3 | 0.3 | 0.3 | 0.3 | 0.3 |
| Preservative | Sodium dehydroacetate | 0.2 | 0.2 | 0.2 | 0.2 | 0.2 | 0.2 | 0.2 | 0.2 | 0.2 | 0.2 |
| Water | Refined water + water in *1 | 63.4 | 63.4 | 63.4 | 63.4 | 65.9 | 61.4 | 71.6 | 61.1 | 70.75 | 57.8 |
| Viscosity (mPa · s) | Shear rate: 3.83 (s⁻¹) | 6 | 7 | 7 | 6 | 8 | 9 | 9 | 6 | 8 | 8 |
| Average particle diameter (nm) | | 157 | 172 | 161 | 168 | 231 | 207 | 288 | 151 | 249 | 274 |
| Adhesion property | | ⊚ | ⊚ | ⊚ | ⊚ | ◯ | ⊚ | ◯ | ◯ | ◯ | ◯ |
| Applying performance | | ⊚ | ⊚ | ⊚ | ⊚ | ◯ | ◯ | ◯ | ◯ | ◯ | ◯ |
| Content liquid stability | | ◯ | ◯ | ◯ | ◯ | ◯ | ◯ | Δ | ◯ | Δ | ◯ |
| Restirring property of | | ◯ | ◯ | ◯ | ◯ | ◯ | ◯ | ◯ | ◯ | ◯ | ◯ |
| precipitated pigment | | | | | | | | | | | |

*1: copolymer emulsion comprising components of acrylic acid, methacrylic acid or (C1 to C4 and C8) alkyl ester thereof

TABLE 2
(total amount: 100% by mass)

| | | Comparative Example | | | | | | | | | | |
|---|---|---|---|---|---|---|---|---|---|---|---|---|
| Component type | Specific compound name | 1 | 2 | 3 | 4 | 5 | 6 | 7 | 8 | 9 | 10 | 11 |
| Pigment | Black iron oxide (spherical | 31 | 10 | | 10 | 10 | 10 | 10 | 10 | 10 | 10 | 10 |
| | particle) | | | | | | | | | | | |
| Pigment | Black iron oxide | | | 10 | | | | | | | | |
| | (octahedral particle) | | | | | | | | | | | |
| Dispersant | Alkyl acrylate copolymer | 5 | 0.4 | 6 | 3 | 3 | 3 | 3 | | | 3 | 3 |
| Dispersant | Octylacrylamide•acrylate | | | | | | | | 3 | 3 | | |
| | copolymer | | | | | | | | | | | |
| Surfactant | Polyoxyethylene behenyl | 3 | 2 | 2 | | | 0.05 | 3.5 | 2 | 2 | 2 | 2 |
| | ether | | | | | | | | | | | |
| Surfactant | Polyethylene glycol | | | | 2 | 2 | | | | | | |
| | monostearate | | | | | | | | | | | |
| Film-forming agent emulsion | Alkyl acrylate copolymer | 20 | 20 | 20 | 4.5 | 36 | 20 | 20 | 20 | 20 | 20 | 20 |
| (resin content in emulsion) | Emulsion *1 | 8.4 | 8.4 | 8.4 | 1.89 | 15.12 | 8.4 | 8.4 | 8.4 | 8.4 | 8.4 | 8.4 |
| Neutralizer | Aminomethylpropanol | 0.3 | 0.3 | 0.3 | 0.3 | 0.3 | 0.3 | 0.3 | 0.3 | 0.3 | 0.3 | 0.3 |
| Volatile alcohol | Phenoxyethanol | 0.4 | 0.4 | 0.4 | 0.4 | 0.4 | 0.4 | 0.4 | 0.04 | 6 | 0.4 | 0.4 |
| Glycols | 1,3-Butylene glycol | 12 | 12 | 12 | 12 | 12 | 12 | 12 | 12 | 12 | 4 | 21 |
| Preservative | Methylparaben | 0.3 | 0.3 | 0.3 | 0.3 | 0.3 | 0.3 | 0.3 | 0.3 | 0.3 | 0.3 | 0.3 |
| Preservative | Sodium dehydroacetate | 0.2 | 0.2 | 0.2 | 0.2 | 0.2 | 0.2 | 0.2 | 0.2 | 0.2 | 0.2 | 0.2 |
| Water | Refined water + water in *1 | 39.4 | 66 | 60.4 | 69.91 | 56.68 | 65.35 | 61.9 | 63.76 | 57.8 | 71.4 | 54.4 |
| Viscosity (mPa · s) | Shear rate: 3.83 (s⁻¹) | 34 | 21 | 16 | 7 | 7 | 18 | 6 | 11 | 9 | 10 | 16 |
| Average particle diameter (nm) | | 292 | 527 | 156 | 172 | 168 | 401 | 162 | 312 | 288 | 318 | 321 |
| Adhesion property | | Δ | Δ | ◯ | X | ⊚ | ◯ | X | ◯ | ◯ | ◯ | ◯ |
| Applying performance | | X | X | X | ◯ | X | X | ◯ | Δ | Δ | Δ | Δ |
| Content liquid stability | | X | X | Δ | ◯ | ◯ | X | ◯ | X | X | X | X |
| Restirring property of | | Δ | X | Δ | ◯ | ◯ | X | ◯ | X | Δ | X | Δ |
| precipitated pigment | | | | | | | | | | | | |

*1: copolymer emulsion comprising components of acrylic acid, methacrylic acid or (C1 to C4 and C8) alkyl ester thereof

As apparent from the results shown in Table 1 and Table 2 described above, it has become clear that the liquid cosmetics prepared in Examples 1 to 10 falling in the scope of the present invention are excellent in an adhesion property, an applying performance, a restirring property of precipitated pigment and a content liquid stability thereof as compared with those prepared in Comparative Examples 1 to 11 falling outside the scope of the present invention.

To observe individually the comparative examples, in Comparative Example 1, the pigment is blended in excess; in Comparative Examples 2 and 3, an amount of the dispersant is out of the scope of the present invention; in Comparative Examples 4 and 5, an amount of the film-forming agent is out of the scope of the present invention; in Comparative Examples 6 and 7, an amount of the surfactant is out of the scope of the present invention; in Comparative Examples 8 and 9, an amount of the volatile alcohol is out of the scope of the present invention; and in Comparative Examples 10 and 11, an amount of the glycols is out of the scope of the present invention. It has been found that in the above cases, the effects of the present invention cannot be exerted.

According to the present invention, obtained is a liquid cosmetic stored in a liquid cosmetic applicator suited to use in an eyeliner or an eyebrow using a brush or a pen feed as an applying means.

What is claimed is:

1. A liquid cosmetic stored in a liquid cosmetic applicator using a brush or a pen feed as an applying means, wherein the above liquid cosmetic comprises at least 1 to 30% by mass of black iron oxide, water, 0.5 to 5% by mass of a dispersant comprising a film-formable resin, 2 to 15% by mass as a solid content of a film-forming agent, 0.1 to 3% by mass of a surfactant, 0.05 to 5% by mass of volatile alcohol, and 5 to 20% by mass of glycols; a viscosity thereof measured at a temperature of 25° C. and a shear rate of 3.83 s⁻¹ by means of an ELD type viscometer falls in a range of 2 to 10 mPa·s; and an observed average particle diameter is 100 to 300 nm.

2. The liquid cosmetic as described in claim 1, wherein the dispersant comprising a film-formable resin is a homopolymer or a copolymer prepared by using one kind or two or more kinds of compounds selected from acrylic acid, methacrylic acid or alkyl esters thereof as a starting material monomer, or a copolymer of at least one of acrylic acid, methacrylic acid or alkyl esters thereof with octylacrylamide.

3. The liquid cosmetic as described in claim 1, wherein the surfactant is a nonionic surfactant.

4. The liquid cosmetic as described in claim 1, wherein the volatile alcohol is phenoxyethanol.

5. The liquid cosmetic as described in claim 1, wherein the glycols are 1,3-butylene glycol.

6. The liquid cosmetic as described in claim 1, wherein the liquid cosmetic applicator is provided with a sliver type vessel in which the liquid cosmetic is held in a sliver or a collector type vessel in which the liquid cosmetic is held in a flat-spread sheet member.

7. The liquid cosmetic as described in claim 1, wherein the liquid cosmetic is used for an eyeliner or an eyebrow.
